(19) United States
(12) Patent Application Publication
(10) Pub. No.: US 2011/0106191 A1
(43) Pub. Date: May 5, 2011

(54) IMPLANTABLE MEDICAL DEVICE NOISE MODE

(75) Inventors: Tommy D. Bennett, Shoreview, MN (US); Paul J. DeGroot, Shoreview, MN (US); Karen Jean Kleckner, New Brighton, MN (US)

(73) Assignee: Medtronic, Inc., Minneapolis, MN (US)

(21) Appl. No.: 12/609,587

(22) Filed: Oct. 30, 2009

(51) Int. Cl. A61N 1/362; A61N 1/39

(52) U.S. Cl. 607/5; 607/27

(57) ABSTRACT

Techniques for activating an alternative operating mode in an implantable medical device based on a determination that the device is within a relatively high noise environment or otherwise exposed to relatively high noise. The implantable medical device can automatically detect its presence in a high noise environment and automatically revert to the alternative operating mode, the device may be manually switched to alternative operating mode, or a hybrid manual/automatic approach may be used to switch the device to alternative operating mode.

### TECHNICAL FIELD

The disclosure relates to medical devices and, more particularly, to minimization of the effects of noise on medical device operation.

### BACKGROUND

A variety of implantable medical devices for delivering a therapy and/or monitoring a physiological condition have been used clinically or proposed for clinical use in patients. Examples include implantable medical devices that deliver therapy to and/or monitor conditions associated with the heart, muscle, nerve, brain, stomach or other organs or tissue. Some implantable medical devices may employ electrodes for the delivery of electrical stimulation to such organs or tissues, electrodes for sensing electrical signals within the patient, which may be generated by such organs or tissue, and/or other sensors for sensing physiological parameters of a patient.

Some implantable medical devices, such as cardiac pacemakers or implantable cardioverter-defibrillators, provide therapeutic electrical stimulation to the heart via electrodes carried by one or more implantable leads. The electrical stimulation may include signals such as pulses or shocks for pacing, cardioversion or defibrillation. In some cases, an implantable medical device may sense intrinsic depolarizations of the heart, and control delivery of stimulation signals to the heart based on the sensed depolarizations. Upon detection of an abnormal rhythm, such as bradycardia, tachycardia or fibrillation, an appropriate electrical stimulation signal or signals may be delivered to restore or maintain a more normal rhythm. For example, in some cases, an implantable medical device may deliver pacing pulses to the heart of the patient upon detecting tachycardia or bradycardia, and deliver cardioversion or defibrillation shocks to the heart upon detecting tachycardia or fibrillation.

In some cases, the sensing of electrical activity within the patient or other physiological parameters of patient by an implantable medical device may be negatively impacted by noise. Noise may include ambient noise, e.g., electromagnetic interference, or noise from the patient, e.g., or noise associated with patient movement, muscle contractions, or electrical myopotentials associated with muscle contractions. For example, the sensing intrinsic depolarizations of the heart by a pacemaker or cardioverter-defibrillator may be negatively impacted by noise. In some cases, the noise may be misidentified by a pacemaker or cardioverter-defibrillator as intrinsic depolarizations of the heart, which may cause the device to incorrectly determine that a tachyarrhythmia is occurring, determine that bradyarrhythmia is not occurring, or otherwise mischaracterize the rhythm of the heart. Ambient noise, such as electromagnetic interference, may be particularly prevalent or a particular concern in an operating room environment, e.g., during surgery to implant or modify an implantable medical device or system that includes an implantable medical device. Various sources of ambient noise, e.g., electromagnetic interference, exist in an operating room environment, such as electrocautery instruments.

To prevent inappropriate detection of a ventricular tachycardia or ventricular fibrillation (VT/VF) in the operating room based on noise in the operating room, surgeons often request (or require) that implantable cardioverter-defibrillators be turned off during the implantation surgery. This course of action generally requires that field service personnel of the manufacturer of the implantable medical device be present at implantation with a full-functionality implantable medical device programmer, which is burdensome to the field service personnel and manufacturer. This course of action may also render the implantable medical device incapable of detecting VT/VF in surgery or if the device is not turned on shortly after the procedure.

### SUMMARY

In general, the disclosure describes techniques for determining that an implantable device is being exposed or will be exposed to a relatively high noise environment or condition and, in response to the determination, adjusting detection of physiological events by the implantable medical device, such as, for example, cardiac events. In this manner, the implantable medical device may avoid mistakenly detecting a physiological event based on the noise and, in some cases, unnecessarily delivering a therapy. An implantable medical device may identify a relatively high noise environment or condition, and revert to an operating mode where it ignores sensed events, or examines sensed events more closely, where it determines that such events are more likely noise signals rather than cardiac events, and/or where it delivers no therapy. The implantable medical device identifies the relatively noisy environment or condition based on a noise mode indication received from a user and/or an external device, which may be less featured than a programmer, and/or based on an analysis of the signals used to sense physiological events. The implantable device may then revert back to its normal operating mode upon user command, cessation of the original noise mode indication, at a predefined time (e.g., end of a surgery), or upon recognizing return to a relatively low noise environment.

In one example, the disclosure is directed to a method comprising receiving in an implantable medical device a signal from an external control device, wherein the signal comprises an instruction from a user to the implantable medical device to revert to a predetermined operating mode for a noise condition, and reverting the implantable medical device to the predetermined operating mode in response to the signal, wherein reverting to the predetermined operating mode comprises modifying at least one of physiological event detection or delivery of therapy in response to physiological event detection by the implantable medical device.

In another example, the disclosure is directed to a system comprising an implantable medical device, wherein the implantable medical device comprises an electrical sensing module for detection of physiological events, circuitry that receives a signal from an external control device, wherein the signal comprises an instruction from a user to the implantable medical device to revert to a predetermined operating mode for a noise condition, and a processor that reverts the implantable medical device to the predetermined operating mode in response to the signal by at least modifying at least one of physiological event detection or delivery of therapy by the implantable medical device in response to physiological event detection by the implantable medical device.

In another example, the disclosure is directed to a system comprising means for receiving in an implantable medical device a signal from an external control device, wherein the signal comprises an instruction from a user to the implantable medical device to revert to a predetermined operating mode for a noise condition, and means for reverting the implantable medical device to the predetermined operating mode in response to the signal, wherein the means for reverting to the predetermined operating mode comprises means for modifying at least one of physiological event detection or delivery of therapy in response to physiological event detection by the implantable medical device.

In another example, the disclosure is directed to a computer readable storage medium comprising instructions that cause a processor of an implantable medical device to receive a signal from an external control device, wherein the signal comprises an instruction from a user to the implantable medical device to revert to a predetermined operating mode for a noise condition, and revert the implantable medical device to the predetermined operating mode in response to the signal, wherein the instructions that cause the processor to reverting to the predetermined operating mode comprise instructions that cause the processor to modify at least one of physiological event detection or delivery of therapy in response to physiological event detection by the implantable medical device.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a conceptual diagram illustrating an example medical system comprising an implantable medical device (IMD) for sensing electrical signals of and delivering stimulation therapy to a heart of a patient via implantable leads.

FIG. 2 is a conceptual diagram further illustrating the IMD and leads of the system of FIG. 1 in conjunction with the heart.

FIG. 3 is a conceptual diagram illustrating another example medical system comprising the IMD of FIG. 1 coupled to a different configuration of leads.

FIG. 4 is a functional block diagram illustrating an example configuration of the IMD of FIG. 1.

FIG. 5 is a functional block diagram illustrating an example electrical sensing module of the IMD of FIG. 1.

FIG. 6A is a functional block diagram of an example configuration of the external programmer shown in FIG. 1, which facilitates user communication with an IMD.

FIG. 6B is a functional block diagram of an example configuration of the external noise mode indicator shown in FIG. 1, which facilitates user communication with an IMD.

FIG. 7 is a block diagram illustrating an example system that includes an external device, such as a server, and one or more computing devices that are coupled to the IMD and programmer and noise mode indicator shown in FIG. 1 via a network.

FIG. 8 is a flow diagram of an example method of operating an implantable medical device in an alternative operating mode when a relatively high level of noise is indicated.

### DETAILED DESCRIPTION

In general, the disclosure describes techniques for operating an implantable medical device (IMD) during a period when the IMD experiences or is exposed to a relatively high level of noise, e.g., ambient noise or noise generated by the patient, which may be interpreted as physiological events of interest, e.g., cardiac depolarizations or other cardiac events, if not recognized as noise. In one example, when the IMD is in an operating room, several devices in the operating room may emit interfering signals that may be detected by the IMD as noise. Such devices may be, for example, x-ray or MRI machines that create fields, or devices used in electrocautery, etc. The interfering signals may be detected as physiological events, such as cardiac or neurological events. In the case of an IMD that monitors cardiac events, the IMD may misinterpret the interfering signals to be cardiac depolarizations, misidentify VT or VF based on the misinterpreted signals, and trigger a therapy (e.g., shock) to remedy the falsely detected condition.

In examples of the disclosure described herein, the IMD may operate in an alternative mode while a relatively high level of noise is present. A user (e.g., physician or clinician) may instruct the IMD to operate in the alternative operating mode, so that certain events are processed differently to determine whether they are false signals or actual cardiac events, e.g., while the IMD is in an environment that is known to have a high level of interfering signals, such as an operating room. In one example, when a user instructs the IMD to operate in the alternative operating mode, the IMD may be placed in a “time out” function, where events that may be processed during normal operation are disregarded, until there is an indication that the IMD need not operate in the alternative operating mode anymore. In an example, the indication may be triggered by an instruction from the user for the IMD to revert back to the normal operating mode or the IMD sensing removal from the relatively high noise environment.

In examples of the disclosure, the IMD may operate in the alternative operating mode until the IMD receives another signal from the user and/or external device, detects cessation of the initial signal from the user and/or device, or detects a reduction in the noise. The alternative mode of operation may be a “sleep mode” where the operation of the IMD is temporarily suspended. In other examples, the alternative operating mode may be a mode in which certain functionality of the IMD, such as detection of physiological events of interest and/or delivery of responsive therapy, is disabled. In another example, during the alternative operating mode, the IMD may revert to a scheme where certain events are assumed to be false signals so that when operating in a “noisy” environment, the IMD may run an algorithm that favors decisions based on an assumption of noise input. In another example, during the alternative operating mode, the IMD may modify the detection of physiological events of interest, such as modifying a number of intervals to detect (NID) for detection of tachyarrhythmia in the case of an IMD configured to detect and treat tachyarrhythmia.

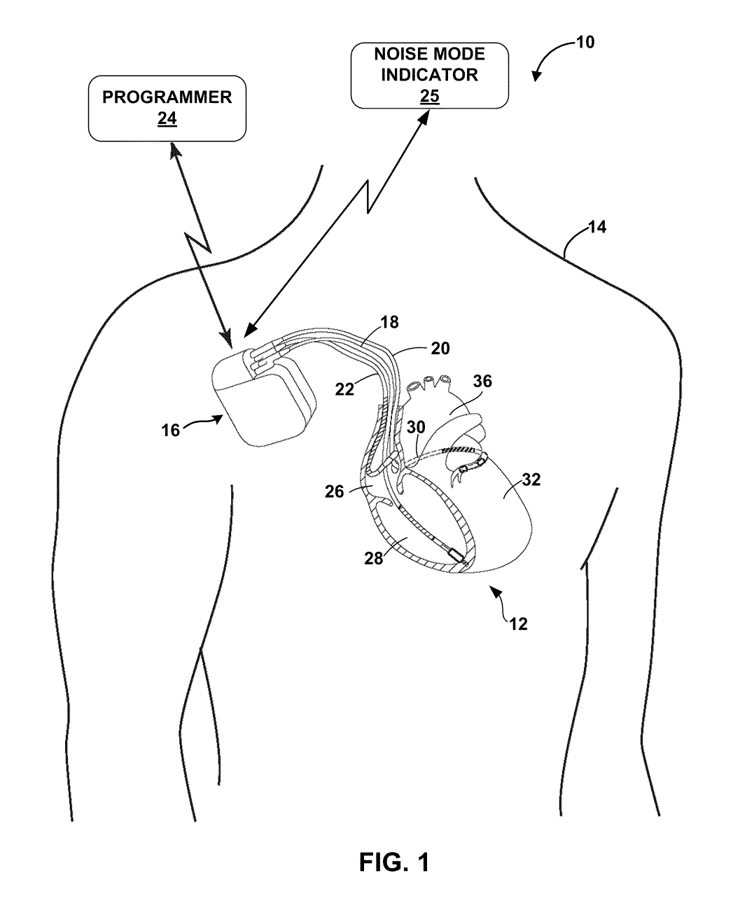

FIG. 1 is a conceptual diagram illustrating an example system 10 that may be used for sensing of physiological parameters of patient 14 and/or to provide therapy to heart 12 of patient 14. Therapy system 10 includes IMD 16, which is coupled to leads 18, 20, and 22, programmer 24, and noise mode indicator 25. System 10 may also include one or more sensors, e.g., in wired or wireless communication with IMD 16. IMD 16 may be, for example, an implantable pacemaker, cardioverter, and/or defibrillator that senses electrical signals of heart 12 and provides electrical signals to heart 12 via electrodes coupled to one or more of leads 18, 20, and 22. Patient 14 is ordinarily, but not necessarily, a human patient.

Although an IMD that delivers electrical stimulation to heart 12 is described herein as an example, the techniques for operating in a noise mode described in this disclosure may be applicable to other IMDs and/or other therapies. In general, the techniques described in this disclosure may be implemented by any IMD that senses physiological events based on electrical signals, where such physiological event-sensing may be impacted by noise, or any one or more components of a system including such an IMD. As one alternative example, the techniques described herein may be implemented by a system that includes an IMD that monitors one or more signals from heart 12 of patient 14, but may not deliver a therapy to heart 12 or patient 14.

As another example, the techniques described herein may be implemented by a system that includes an implantable neurostimulator that delivers electrical stimulation to and/or monitors conditions associated with the brain, spinal cord, or neural tissue of patient 14, instead of or in addition to a cardiac IMD.

Leads 18, 20, 22 extend into the heart 12 of patient 16 to sense electrical activity of heart 12 and/or deliver electrical stimulation to heart 12. In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into the coronary sinus 30 to a region adjacent to the free wall of left ventricle 32 of heart 12. Right atrial (RA) lead 22 extends through one or more veins and the vena cava, and into the right atrium 26 of heart 12. In some alternative examples, system 10 may include an additional lead or lead segment (not shown in FIG. 1) that deploys one or more electrodes within the vena cava or other vein. These electrodes may allow alternative electrical sensing configurations that may provide improved sensing accuracy in some patients. In other examples, an IMD need not be coupled to leads, and instead may sense electrical signals via a plurality of electrodes formed on or integral with a housing of the IMD.

IMD 16 may sense electrical signals attendant to the depolarization and repolarization of heart 12 via electrodes (not shown in FIG. 1) coupled to at least one of the leads 18, 20, 22. In some examples, IMD 16 provides pacing pulses to heart 12 based on the electrical signals sensed within heart 12. The configurations of electrodes used by IMD 16 for sensing and pacing may be unipolar or bipolar. IMD 16 may also provide defibrillation therapy and/or cardioversion therapy via electrodes located on at least one of the leads 18, 20, 22. IMD 16 may detect arrhythmia of heart 12, such as fibrillation of ventricles 28 and 32, and deliver cardioversion or defibrillation therapy to heart 12 in the form of electrical pulses. In some examples, IMD 16 may be programmed to deliver a progression of therapies, e.g., pulses with increasing energy levels, until a tachyarrhythmia of heart 12 is stopped. IMD 16 detects tachycardia or fibrillation employing one or more tachycardia or fibrillation detection techniques known in the art.

In some examples, programmer 24 may be a handheld computing device, computer workstation, or networked computing device. Programmer 24 may include a user interface that receives input from a user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 24 can additionally or alternatively include a peripheral pointing device, such as a mouse, via which a user may interact with the user interface. In some examples, a display of programmer 24 may include a touch screen display, and a user may interact with programmer 24 via the display. It should be noted that the user may also interact with programmer 24 or IMD 16 remotely via a networked computing device.

A user, such as a physician, technician, surgeon, electrophysiologist, or other clinician, may interact with programmer 24 to communicate with IMD 16. For example, the user may interact with programmer 24 to retrieve physiological or diagnostic information from IMD 16. A user may also interact with programmer 24 to program IMD 16, e.g., select values for operational parameters of the IMD 16.

For example, the user may use programmer 24 to retrieve information from IMD 16 regarding the rhythm of heart 12, trends therein over time, or arrhythmic episodes. As another example, the user may use programmer 24 to retrieve information from IMD 16 regarding other sensed physiological parameters of patient 14, such as intracardiac or intravascular pressure, activity, posture, respiration, or thoracic impedance. As another example, the user may use programmer 24 to retrieve information from IMD 16 regarding the performance or integrity of IMD 16 or other components of system 10, such as leads 18, 20 and 22, or a power source of IMD 16.

IMD 16 and programmer 24 may communicate via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the patient's body near the IMD 16 implant site in order to improve the quality or security of communication between IMD 16 and programmer 24.

In some examples, noise mode indicator 25 may be a handheld computing device. In some examples, noise mode indicator 25 is less fully-featured with respect to its ability to control or interact with IMD 16 and/or a user than programmer 24. In some examples, noise mode indicator 25 may be limited to providing a signal to IMD 16 that indicates a noisy environment or condition, in which case IMD 16 may respond to the signal by entering operational mode suitable for a noisy environment or condition, as described herein. In other examples, noise mode indicator 25 is preconfigured to provide one or more instructions to IMD 16 that change operational parameters of the IMD to place the IMD in the noise mode, e.g., without requiring a user to input commands for the various instructions. Noise mode indicator 25 may receive a user command to provide the indication or instructions to IMD 16, or may automatically provide such an indication or instructions in response to being powered on.

IMD 16 and noise mode indicator 25 may communicate via wireless communication using any techniques known in the art. Examples of communication techniques may include, for example, low frequency or radiofrequency (RF) telemetry, but other techniques are also contemplated.

In one example, a clinician or a user may use noise mode indicator 25 to send an indication to IMD 16 to operate in a different mode while its surrounding environment is, for example, an operating room or an area where there may be present interfering signals. The indication may be sent by noise mode indicator 25 in response to a press of a key. In some examples, the key is an ON/OFF button, and the indication is sent in response to powering on the noise mode indicator 25. In some examples, the indication is a signal or beacon delivered continuously or periodically by noise mode indicator 25 so long as IMD 16 is to operate in the different mode. In some examples, noise mode indicator 25 provides the indication or signal so long as the noise mode indicator is powered on.

In some examples, IMD 16 reverts to its normal mode of operation in response to cessation of the signal or beacon, or delivery of a second signal or indication by noise mode indicator 25. Cessation of the signal or beacon, or delivery of a second signal or indication, may be in response to another input from a user, which may be a press of the key or another key, e.g., to power down the noise mode indicator

The clinician or user may also program the noise mode indicator 25 to send an indication to the IMD 16 to revert to the alternative operating mode at some point in the future, e.g., for predetermined period of time, when it is known that the patient implanted with the IMD will be in a high noise environment. In a high noise environment, interfering signals may cause episodes or events that may be non-physiological, which may in a normal operating mode be sensed as conditions requiring delivery of therapy by IMD 16. When operating in the alternative operating mode, such interfering signals may be sensed and processed by IMD 16 to determine whether they are conditions for which therapy should be delivered or noise signals caused by the surrounding environment and should therefore be disregarded.

In other examples, one or more devices other than IMD 16 may, alone, or in combination with IMD 16, implement the techniques described herein. For example, noise mode indicator 25 or another external device may store may process the sensed signals to determine whether to deliver therapy or disregard the signals as noise.

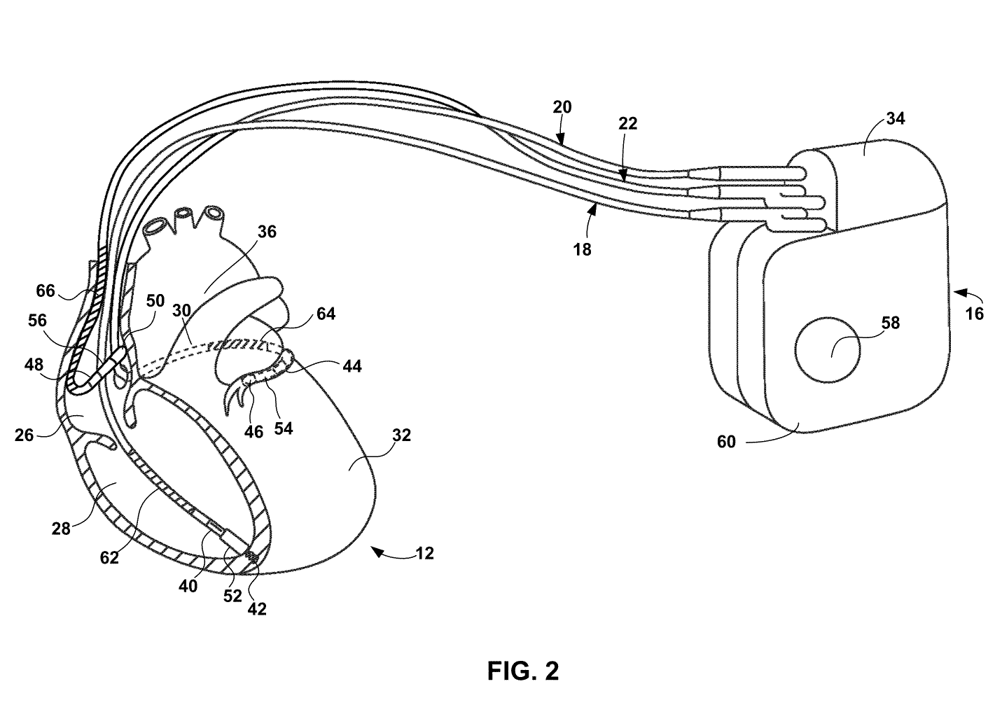

FIG. 2 is a conceptual diagram illustrating a three-lead IMD 16 and leads 18, 20 and 22 of therapy system 10 in greater detail. Leads 18, 20 and 22 may be electrically coupled to a signal generator and a sensing module of IMD 16 via connector block 34. In some examples, proximal ends of leads 18, 20 and 22 may include electrical contacts that electrically couple to respective electrical contacts within connector block 34 of IMD 16. In addition, in some examples, leads 18, 20 and 22 may be mechanically coupled to connector block 34 with the aid of set screws, connection pins, snap connectors, or another suitable mechanical coupling mechanism.

Each of the leads 18, 20 and 22 includes an elongated insulative lead body, which may carry a number of concentric coiled conductors separated from one another by tubular insulative sheaths. Bipolar electrodes 40 and 42 are located adjacent to a distal end of lead 18 in right ventricle 28. In addition, bipolar electrodes 44 and 46 are located adjacent to a distal end of lead 20 in coronary sinus 30 and bipolar electrodes 48 and 50 are located adjacent to a distal end of lead 22 in right atrium 26. There are no electrodes located in left atrium 36 in the illustrated example, but other examples may include electrodes in left atrium 36.

Electrodes 40, 44 and 48 may take the form of ring electrodes, and electrodes 42, 46 and 50 may take the form of extendable helix tip electrodes mounted retractably within insulative electrode heads 52, 54 and 56, respectively. In other examples, one or more of electrodes 42, 46 and 50 may take the form of small circular electrodes at the tip of a tined lead or other fixation element. Leads 18, 20, 22 also include elongated electrodes 62, 64, 66, respectively, which may take the form of a coil. Each of the electrodes 40, 42, 44, 46, 48, 50, 62, 64 and 66 may be electrically coupled to a respective one of the coiled conductors within the lead body of its associated lead 18, 20, 22, and thereby coupled to respective ones of the electrical contacts on the proximal end of leads 18, 20 and 22.

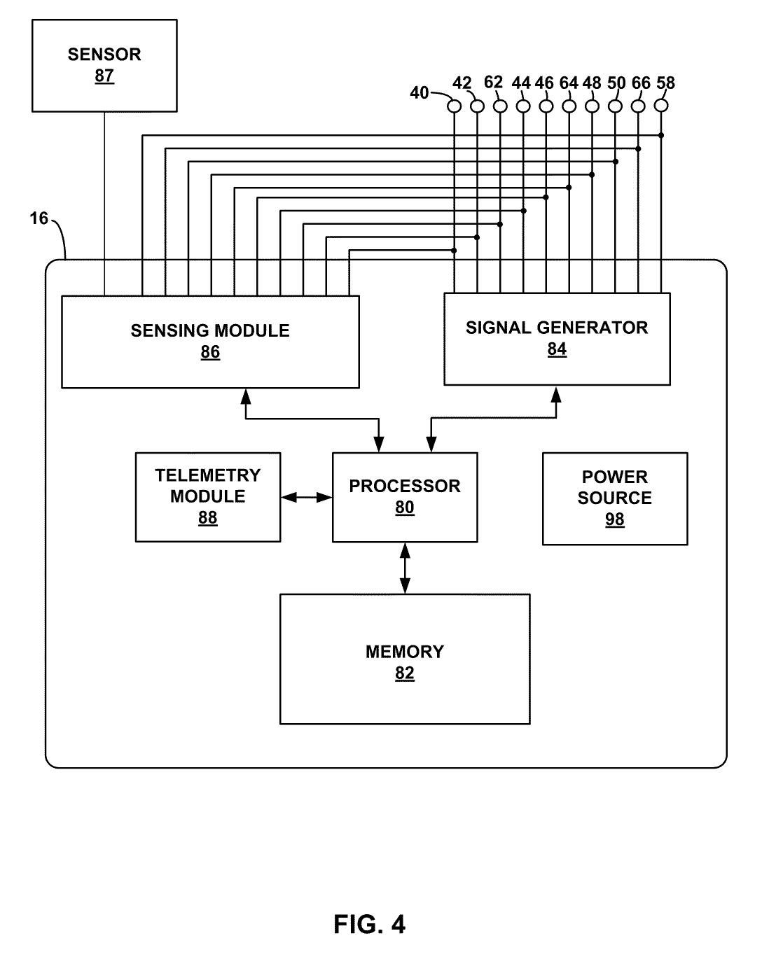

In some examples, as illustrated in FIG. 2, IMD 16 includes one or more housing electrodes, such as housing electrode 58, which may be formed integrally with an outer surface of hermetically-sealed housing 60 of IMD 16 or otherwise coupled to housing 60. In some examples, housing electrode 58 is defined by an uninsulated portion of an outward facing portion of housing 60 of IMD 16. Other division between insulated and uninsulated portions of housing 60 may be employed to define two or more housing electrodes. In some examples, housing electrode 58 comprises substantially all of housing 60. As described in further detail with reference to FIG. 4, housing 60 may enclose a signal generator that generates therapeutic stimulation, such as cardiac pacing pulses and defibrillation shocks, as well as a sensing module for monitoring the rhythm of heart 12.

IMD 16 may sense electrical signals attendant to the depolarization and repolarization of heart 12 via electrodes 40, 42, 44, 46, 48, 50, 62, 64 and 66. The electrical signals are conducted to IMD 16 from the electrodes via the respective leads 18, 20, 22. IMD 16 may sense such electrical signals via any bipolar combination of electrodes 40, 42, 44, 46, 48, 50, 62, 64 and 66. Furthermore, any of the electrodes 40, 42, 44, 46, 48, 50, 62, 64 and 66 may be used for unipolar sensing in combination with housing electrode 58. The combination of electrodes used for sensing may be referred to as a sensing configuration.

In some examples, IMD 16 delivers pacing pulses via bipolar combinations of electrodes 40, 42, 44, 46, 48 and 50 to produce depolarization of cardiac tissue of heart 12. In some examples, IMD 16 delivers pacing pulses via any of electrodes 40, 42, 44, 46, 48 and 50 in combination with housing electrode 58 in a unipolar configuration. Furthermore, IMD 16 may deliver defibrillation pulses to heart 12 via any combination of elongated electrodes 62, 64, 66, and housing electrode 58. Electrodes 58, 62, 64, 66 may also be used to deliver cardioversion pulses to heart 12. Electrodes 62, 64, 66 may be fabricated from any suitable electrically conductive material, such as, but not limited to, platinum, platinum alloy or other materials known to be usable in implantable defibrillation electrodes.

The configuration of therapy system 10 illustrated in FIGS. 1 and 2 is merely one example. In other examples, a therapy system may include epicardial leads, patch electrodes, and/or subcutaneous electrodes instead of or in addition to the transvenous leads 18, 20, 22 illustrated in FIG. 1. In some examples, IMD 16 need not be coupled to leads, and may instead sense cardiac electrical signals via a plurality of electrodes formed on or integrally with the housing of the IMD. Further, IMD 16 need not be implanted within patient 14. In examples in which IMD 16 is not implanted in patient 14, IMD 16 may deliver defibrillation pulses and other therapies to heart 12 via percutaneous leads that extend through the skin of patient 14 to a variety of positions within or outside of heart 12.

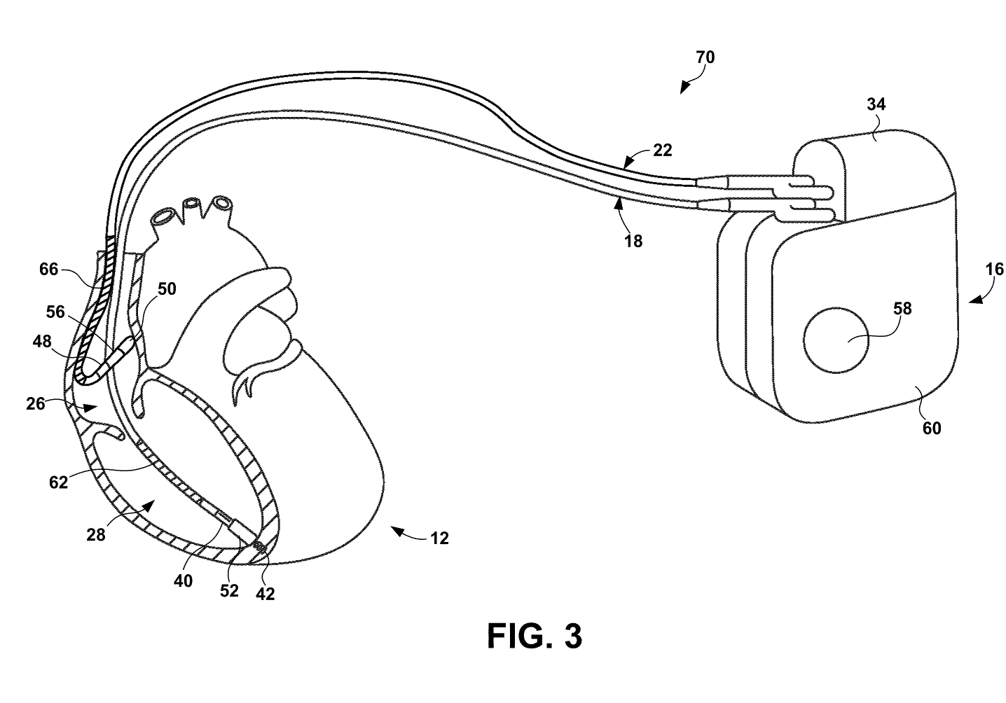

In addition, in other examples, a therapy system may include any suitable number of leads coupled to IMD 16, and each of the leads may extend to any location within or proximate to heart 12. For example, other examples of therapy systems may include three transvenous leads located as illustrated in FIGS. 1 and 2, and an additional lead located within or proximate to left atrium 36. As another example, other examples of therapy systems may include a single lead that extends from IMD 16 into right atrium 26 or right ventricle 28, or two leads that extend into a respective one of the right ventricle 26 and right atrium 26. An example of this type of therapy system is shown in FIG. 3. Any electrodes located on these additional leads may be used in sensing and/or stimulation configurations.

FIG. 3 is a conceptual diagram illustrating another example system 70, which is similar to system 10 of FIGS. 1 and 2, but includes two leads 18, 22, rather than three leads. Leads 18, 22 are implanted within right ventricle 28 and right atrium 26, respectively. System 70 shown in FIG. 3 may be useful for sensing cardiac electrical signals, providing defibrillation and pacing pulses to heart 12, and distinguishing between signals indicating cardiac events and noise signals to correctly determine whether to provide pulses to the heart, as described herein.

FIG. 4 is a functional block diagram illustrating an example configuration of IMD 16. In the illustrated example, IMD 16 includes a processor 80, memory 82, signal generator 84, sensing module 86, telemetry module 88, and power source 90. Memory 82 includes computer-readable instructions that, when executed by processor 80, cause IMD 16 and processor 80 to perform various functions attributed to IMD 16 and processor 80 herein. Memory 82 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media.

Processor 80 may include any one or more of a microprocessor, a controller, a digital signal processor (DSP), an application specific integrated circuit (ASIC), a field-programmable gate array (FPGA), or equivalent discrete or analog logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. The functions attributed to processor 80 herein may be embodied as software, firmware, hardware or any combination thereof.

Processor 80 controls signal generator 84 to deliver stimulation therapy to heart 12 according to a selected one or more of therapy programs, which may be stored in memory 82. For example, processor 80 may control stimulation generator 84 to deliver electrical pulses with the amplitudes, pulse widths, frequency, or electrode polarities specified by the selected one or more therapy programs.

Signal generator 84 is electrically coupled to electrodes 40, 42, 44, 46, 48, 50, 58, 62, 64, and 66, e.g., via conductors of the respective lead 18, 20, 22, or, in the case of housing electrode 58, via an electrical conductor disposed within housing 60 of IMD 16. In the illustrated example, signal generator 84 is configured to generate and deliver electrical stimulation therapy to heart 12. For example, signal generator 84 may deliver defibrillation shocks to heart 12 via at least two electrodes 58, 62, 64, 66. Signal generator 84 may deliver pacing pulses via ring electrodes 40, 44, 48 coupled to leads 18, 20, and 22, respectively, and/or helical electrodes 42, 46, and 50 of leads 18, 20, and 22, respectively. In some examples, signal generator 84 delivers pacing, cardioversion, or defibrillation stimulation in the form of electrical pulses. In other examples, signal generator may deliver one or more of these types of stimulation in the form of other signals, such as sine waves, square waves, or other substantially continuous time signals.

Signal generator 84 may include a switch module and processor 80 may use the switch module to select, e.g., via a data/address bus, which of the available electrodes are used to deliver defibrillation pulses or pacing pulses. The switch module may include a switch array, switch matrix, multiplexer, or any other type of switching device suitable to selectively couple stimulation energy to selected electrodes.

Sensing module 86 monitors signals from at least one of electrodes 40, 42, 44, 46, 48, 50, 58, 62, 64 or 66 in order to monitor electrical activity of heart 12. Sensing module 86 may also include a switch module to select which of the available electrodes are used to sense the heart activity, depending upon which electrode combination is used in the current sensing configuration. In some examples, processor 80 may select the electrodes that function as sense electrodes, i.e., select the sensing configuration, via the switch module within sensing module 86.

Sensing module 86 may include one or more detection channels, each of which may comprise an amplifier. The detection channels may be used to sense the cardiac signals. Some detection channels may detect events, such as R- or P-waves, and provide indications of the occurrences of such events to processor 80. One or more other detection channels may provide the signals to an analog-to-digital converter, for processing or analysis by processor 80. In response to the signals from processor 80, the switch module within sensing module 86 may couple selected electrodes to selected detection channels.

For example, sensing module 86 may comprise one or more narrow band channels, each of which may include a narrow band filtered sense-amplifier that compares the detected signal to a threshold. If the filtered and amplified signal is greater than the threshold, the narrow band channel indicates that a certain electrical cardiac event, e.g., depolarization, has occurred. Processor 80 then uses that detection in measuring frequencies of the sensed events. Different narrow band channels of sensing module 86 may have distinct functions. For example, some various narrow band channels may be used to sense either atrial or ventricular events.

In some examples, sensing module 86 includes a wide band channel which may comprise an amplifier with a relatively wider pass band than the R-wave or P-wave amplifiers. Signals from the selected sensing electrodes that are selected for coupling to this wide-band amplifier may be converted to multi-bit digital signals by an analog-to-digital converter (ADC) provided by, for example, sensing module 86 or processor 80. In some examples, processor 80 may store the digitized versions of signals from the wide band channel in memory 82 as electrograms (EGMs). In some examples, processor 80 may employ digital signal analysis techniques to characterize the digitized signals from the wide band channel to, for example detect and classify the patient's heart rhythm. Processor 80 may detect and classify the patient's heart rhythm by employing any of the numerous signal processing methodologies known in the art.

Sensing module 86 may also include or be coupled to one or more sensors 87 separate from electrodes 40, 42, 44, 46, 48, 50, 58, 62, 64 and 66. For example, one or more sensors 87 may be located within a housing of IMD 16, coupled to IMD 16 via one or more of leads 18, 20 and 22, or may be in wireless communicate with IMD 16. Via a signal generated by sensor 87, processor 80 may monitor one or more physiological parameters, such as blood pressure, blood flow, or patient activity.

Processor 80 may maintain one or more programmable interval counters. If IMD 16 is configured to generate and deliver pacing pulses to heart 12, processor 80 may maintain programmable counters which control the basic time intervals associated with various modes of pacing, including cardiac resynchronization therapy (CRT) and anti-tachycardia pacing (ATP). In examples in which IMD 16 is configured to deliver pacing therapy, intervals defined by processor 80 may include atrial and ventricular pacing escape intervals, refractory periods during which sensed P-waves and R-waves are ineffective to restart timing of the escape intervals, and the pulse widths of the pacing pulses. As another example, processor 80 may define a blanking period, and provide signals to sensing module 86 to blank one or more channels, e.g., amplifiers, for a period during and after delivery of electrical stimulation to heart 12. The durations of these intervals may be determined by processor 80 in response to stored data in memory 82. Processor 80 may also determine the amplitude of the cardiac pacing pulses.

Processor 80 may reset interval counters upon sensing of R-waves and P-waves with detection channels of sensing module 86. For pacing, signal generator 84 may include pacer output circuits that are coupled, e.g., selectively by a switching module, to any combination of electrodes 40, 42, 44, 46, 48, 50, 58, 62, or 66 appropriate for delivery of a bipolar or unipolar pacing pulse to one of the chambers of heart 12. Processor 80 may also reset the interval counters upon the generation of pacing pulses by signal generator 84, and thereby control the basic timing of cardiac pacing functions, including CRT and ATP.

The value of the count present in the interval counters when reset by sensed R-waves and P-waves may be used by processor 80 to measure the durations of R-R intervals, P-P intervals, PR intervals and R-P intervals, which are measurements that may be stored in memory 82. Processor 80 may use the count in the interval counters to detect a tachyarrhythmia event, such as ventricular fibrillation or ventricular tachycardia. In some examples, a portion of memory 82 may be configured as a plurality of recirculating buffers, capable of holding series of measured intervals, which may be analyzed by processor 80 to determine whether the patient's heart 12 is presently exhibiting atrial or ventricular tachyarrhythmia.

In some examples, an arrhythmia detection method may include any suitable tachyarrhythmia detection algorithms. In one example, processor 80 may utilize all or a subset of the rule-based detection methods described in U.S. Pat. No. 5,545,186 to Olson et al., entitled, “PRIORITIZED RULE BASED METHOD AND APPARATUS FOR DIAGNOSIS AND TREATMENT OF ARRHYTHMIAS,” which issued on Aug. 13, 1996, or in U.S. Pat. No. 5,755,736 to Gillberg et al., entitled, “PRIORITIZED RULE BASED METHOD AND APPARATUS FOR DIAGNOSIS AND TREATMENT OF ARRHYTHMIAS,” which issued on May 26, 1998. U.S. Pat. No. 5,545,186 to Olson et al. U.S. Pat. No. 5,755,736 to Gillberg et al. is incorporated herein by reference in their entireties. However, other arrhythmia detection methodologies may also be employed by processor 80 in other examples.

In some examples, processor 80 may determine that tachyarrhythmia has occurred by identification of shortened R-R (or P-P) interval lengths. Generally, processor 80 detects tachycardia when the interval length falls below 360 milliseconds (ms) and fibrillation when the interval length falls below 240 ms. These interval lengths are merely examples, and a user may define the interval lengths as desired, which may then be stored within memory 82. This interval length may need to be detected for a certain number of consecutive cycles, for a certain percentage of cycles within a running window, or a running average for a certain number of cardiac cycles, as examples.

In some examples, processor 80 may analyze the morphology of the digitized signals from wide band channel 104 to distinguish between noise and cardiac depolarization, by comparing the morphology of a signal to templates of known noise patterns corresponding to devices that usually produce the noise signals that may be detected as fast rate events. In some examples, based on morphological analysis, processor 80 may determine whether a sensed signal indicates a cardiac event or noise. In some examples, the fast rate event may be an event that is “new” to the processor and to which there may be no existing template in the stored noise signal templates. In this example, the processor may treat the unknown new fast rate event as noise and disregard it without delivering therapy. Often, noise events are not a random event and would reoccur, therefore, when a new event is sensed, the processor may check again for the source of the noise and attempt to detect the “new” noise signal again. If the “new” noise signal is detected again, the processor may determine to continue operating in the alternative mode. Otherwise, if the “new” noise signal is not detected again, then there may be no noise source, and the processor may determine to return to the normal operating mode.

In some examples according to this disclosure, the morphology of a sensed signal may be compared to templates of physiological signals, and based on morphological analysis, processor 80 may determine a sensed signal indicates noise if there is no match between the sensed signal and the templates of physiological signals. In other examples, the morphology of a sensed signal may be compared to template of physiological signals and to templates of known noise patterns corresponding to devices that usually produce the noise signals that may be detected as fast rate events, to determine which template may be the closest match to the sensed signal, or to determine that the sensed signal may be a “new” noise signal.

In some examples according to this disclosure, processor 80 may revert while in a highly noisy environment, e.g., during a medical procedure, to an alternative operating mode. In such an environment, noise or other interfering signals may be, in a normal operating mode, falsely interpreted as cardiac events, e.g., depolarization or VT/VF, triggering a delivery of an unnecessary shock to the heart. When processor 80 reverts to the alternative operating mode, an alternative algorithm may be used to assess whether a signal is a cardiac event or noise, and provide appropriate shock therapy if it is a cardiac event, and disregard the signal if it is merely noise.

In some examples, processor 80 may automatically revert to the alternative operating mode, when it senses the presence of additional outside signal sources such as, for example, machines in an operating room that create interfering fields. Processor 80, subsequently, may revert back to normal operating mode when the interfering machines may be out of range, for example, when a patient with IMD 16 is taken out of the operating room. Processor 80 may identify the presence and absence of the interfering signals from one or more other sensing channels or sensors 87. In other examples, processor 80 may automatically revert back to the normal operating mode after a predetermined period of time.

In other examples, processor 80 may revert to the alternative operating mode when a user input instructs the processor to change modes. The user input may be, for example, a selection on the user interface of a noise mode indicator 25. In some examples, the user may activate the alternative operating mode by entering a single command via noise mode indicator 25, such as depression of a single key or combination of keys of a keypad or a single point-and-select action with a pointing device. The user may subsequently use the same user input to instruct the processor 80 to revert back to normal operating mode. The user selection may be communicated from the noise mode indicator 25 to the processor 80 via telemetry module 88.

Telemetry module 88 includes any suitable hardware, firmware, software or any combination thereof for communicating with other devices, such as programmer 24 and noise mode indicator 25 (FIG. 1). Under the control of processor 80, telemetry module 88 may receive downlink telemetry from and send uplink telemetry to programmer 24 with the aid of an antenna, which may be internal and/or external. Processor 80 may provide the data to be uplinked to programmer 24 and the control signals for the telemetry circuit within telemetry module 88, e.g., via an address/data bus. In some examples, telemetry module 88 may provide received data to processor 80 via a multiplexer.

The various components of IMD 16 are coupled to power source 98, which may include a rechargeable or non-rechargeable battery. A non-rechargeable battery may be capable of holding a charge for several years, while a rechargeable battery may be inductively charged from an external device, e.g., on a daily or weekly basis.

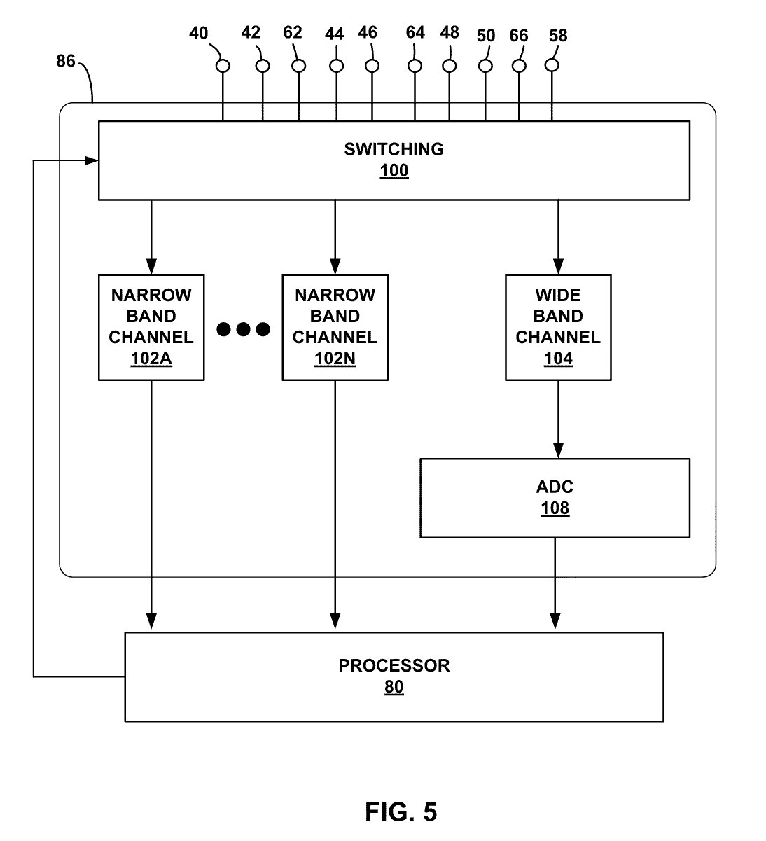

FIG. 5 is a block diagram of an example configuration of electrical sensing module 86. As shown in FIG. 5, electrical sensing module 86 includes multiple components including a switching module 100, narrow band channels 102A to 102N (collectively “narrow band channels 102”), wide band channel(s) 104, and analog to digital converter (ADC) 108. Switching module 100 may, based on control signals from processor 80, control which of electrodes 40, 42, 44, 46, 48, 50, 58, 62, 64 and 66 is coupled to which of channels 102 and 104 at any given time.

Each of narrow band channels 102 may comprise a narrow band filtered sense-amplifier that compares the detected signal to a threshold. If the filtered and amplified signal is greater than the threshold, the narrow band channel indicates that a certain electrical heart event has occurred. Processor 80 then uses that detection in measuring frequencies of the detected events. Narrow band channels 102 may have distinct functions. For example, some various narrow band channels may be used to detect either atrial or ventricular events.

In one example, at least one narrow band channel 102 may include an R-wave amplifier that receives signals from the sensing electrode configuration of electrodes 40 and 42, which are used for sensing and/or pacing in right ventricle 28 of heart 12. Another narrow band channel 102 may include another R-wave amplifier that receives signals from the sensing electrode configuration of electrodes 44 and 46, which are used for sensing and/or pacing proximate to left ventricle 32 of heart 12. In some examples, the R-wave amplifiers may take the form of an automatic gain controlled amplifier that provides an adjustable sensing threshold as a function of the measured R-wave amplitude of the heart rhythm.

In addition, in some examples, a narrow band channel 102 may include a P-wave amplifier that receives signals from electrodes 48 and 50, which are used for pacing and sensing in right atrium 26 of heart 12. In some examples, the P-wave amplifier may take the form of an automatic gain controlled amplifier that provides an adjustable sensing threshold as a function of the measured P-wave amplitude of the heart rhythm. Examples of R-wave and P-wave amplifiers are described in U.S. Pat. No. 5,117,824 to Keimel et al., which issued on Jun. 2, 1992 and is entitled, “APPARATUS FOR MONITORING ELECTRICAL PHYSIOLOGIC SIGNALS,” and is incorporated herein by reference in its entirety. Other amplifiers may also be used. Furthermore, in some examples, one or more of the sensing channels of sensing module 86 may be selectively coupled to housing electrode 58, or elongated electrodes 62, 64, or 66, with or instead of one or more of electrodes 40, 42, 44, 46, 48 or 50, e.g., for unipolar sensing of R-waves or P-waves in any of chambers 26, 28, or 32 of heart 12.

Wide band channel 104 may comprise an amplifier with a relatively wider pass band than the R-wave or P-wave amplifiers. Signals from the sensing electrode configuration that is selected for coupling to this wide-band amplifier may be converted to multi-bit digital signals by ADC 108. In some examples, processor 80 may store signals the digitized versions of signals from wide band channel 104 in memory 82 as EGMs. In some examples, the storage of such EGMs in memory 82 may be under the control of a direct memory access circuit.

In some examples, processor 80 may employ digital signal analysis techniques to characterize the digitized signals from wide band channel 104 to, for example detect and classify the patient's heart rhythm. Processor 80 may detect and classify the patient's heart rhythm by employing any of the numerous signal processing methodologies known in the art. Further, in some examples, processor 80 may analyze the morphology of the digitized signals from wide band channel 104 to distinguish between noise and cardiac depolarizations.

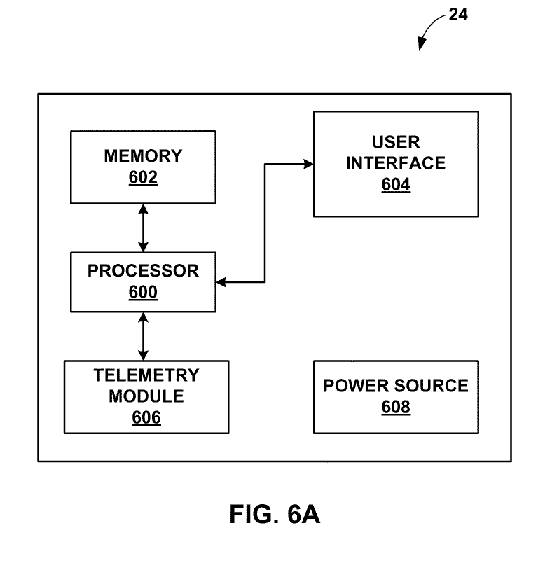

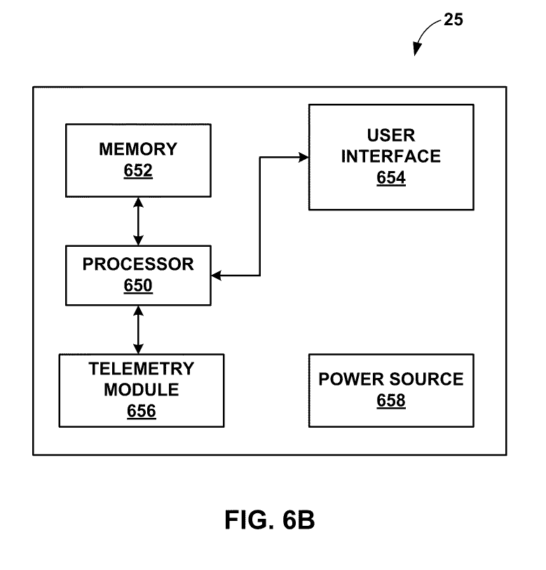

FIG. 6A is functional block diagram illustrating an example configuration of programmer 24. As shown in FIG. 6A, programmer 24 may include a processor 600, memory 602, user interface 604, telemetry module 606, and power source 608. Programmer 24 may be a dedicated hardware device with dedicated software for programming of IMD 16. Alternatively, programmer 24 may be an off-the-shelf computing device running an application that enables programmer 24 to program IMD 16.

A user may use programmer 24 to select therapy programs (e.g., sets of stimulation parameters), generate new therapy programs, modify therapy programs through individual or global adjustments or transmit the new programs to a medical device, such as IMD 16 (FIG. 1). The clinician may interact with programmer 24 via user interface 104, which may include display to present graphical user interface to a user, and a keypad or another mechanism for receiving input from a user.

Processor 600 can take the form one or more microprocessors, DSPs, ASICs, FPGAs, programmable logic circuitry, or the like, and the functions attributed to processor 600 herein may be embodied as hardware, firmware, software or any combination thereof. Memory 602 may store instructions that cause processor 600 to provide the functionality ascribed to programmer 24 herein, and information used by processor 600 to provide the functionality ascribed to programmer 24 herein. Memory 602 may include any fixed or removable magnetic, optical, or electrical media, such as RAM, ROM, CD-ROM, hard or floppy magnetic disks, EEPROM, or the like. Memory 602 may also include a removable memory portion that may be used to provide memory updates or increases in memory capacities. A removable memory may also allow patient data to be easily transferred to another computing device, or to be removed before programmer 24 is used to program therapy for another patient.

Programmer 24 may communicate wirelessly with IMD 16, such as using RF communication or proximal inductive interaction. This wireless communication is possible through the use of telemetry module 656, which may be coupled to an internal antenna or an external antenna. An external antenna that is coupled to programmer 24 may correspond to the programming head that may be placed over heart 12, as described above with reference to FIG. 1. Telemetry module 606 may be similar to telemetry module 88 of IMD 16 (FIG. 4).

Telemetry module 606 may also be configured to communicate with another computing device via wireless communication techniques, or direct communication through a wired connection. Examples of local wireless communication techniques that may be employed to facilitate communication between programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 24 without needing to establish a secure wireless connection. An additional computing device in communication with programmer 24 may be a networked device such as a server capable of processing information retrieved from IMD 16.

Power source 608 delivers operating power to the components of programmer 24. Power source 608 may include a battery and a power generation circuit to produce the operating power. In some examples, the battery may be rechargeable to allow extended operation. Recharging may be accomplished by electrically coupling power source 608 to a cradle or plug that is connected to an alternating current (AC) outlet. In addition or alternatively, recharging may be accomplished through proximal inductive interaction between an external charger and an inductive charging coil within programmer 24. In other examples, traditional batteries (e.g., nickel cadmium or lithium ion batteries) may be used. In addition, programmer 24 may be directly coupled to an alternating current outlet to power programmer 24. Power source 608 may include circuitry to monitor power remaining within a battery. In this manner, user interface 604 may provide a current battery level indicator or low battery level indicator when the battery needs to be replaced or recharged. In some cases, power source 608 may be capable of estimating the remaining time of operation using the current battery.

FIG. 6B is functional block diagram illustrating an example configuration of noise mode indicator 25. As shown in FIG. 6B, noise mode indicator 25 may include a processor 650, memory 652, user interface 654, telemetry module 656, and power source 658. Noise mode indicator 25 may be a dedicated hardware device with dedicated software for programming of IMD 16. Alternatively, noise mode indicator 25 may be an off-the-shelf computing device running an application that enables noise mode indicator 25 to program IMD 16.

The user may use noise mode indicator 25 to instruct IMD 16 to revert to an alternative operating mode with higher sensitivity to noise. Alternatively, noise mode indicator 25 may be used by a clinician or a user to schedule a future period during which the IMD 16 should revert to an alternative operating mode.

Processor 650 can take the form one or more microprocessors, DSPs, ASICs, FPGAs, programmable logic circuitry, or the like, and the functions attributed to processor 650 herein may be embodied as hardware, firmware, software or any combination thereof. Memory 652 may store instructions that cause processor 650 to provide the functionality ascribed to noise mode indicator 25 herein, and information used by processor 650 to provide the functionality ascribed to noise mode indicator 25 herein. Memory 652 may include any fixed or removable magnetic, optical, or electrical media, such as RAM, ROM, CD-ROM, hard or floppy magnetic disks, EEPROM, or the like.

Noise mode indicator 25 may communicate wirelessly with IMD 16, such as using RF communication or proximal inductive interaction. This wireless communication is possible through the use of telemetry module 656, which may be coupled to an internal antenna or an external antenna. Telemetry module 656 may be similar to telemetry module 88 of IMD 16 (FIG. 4).

Telemetry module 656 may also be configured to communicate with another computing device via wireless communication techniques, or direct communication through a wired connection. Examples of local wireless communication techniques that may be employed to facilitate communication between noise mode indicator 25 and another computing device include RF communication according to the 802.11 or Bluetooth specification sets, infrared communication, e.g., according to the IrDA standard, or other standard or proprietary telemetry protocols. In this manner, other external devices may be capable of communicating with noise mode indicator 25 without needing to establish a secure wireless connection. An additional computing device in communication with noise mode indicator 25 may be a networked device such as a server capable of processing information retrieved from IMD 16.

In some examples, IMD 16 and noise mode indicator 25 may be configured to communicate via other wireless communication techniques, in addition to or instead of RF communication. For example, IMD 16 and noise mode indicator 25 may be configured to communicate ultrasonically or vibrationally. As another example, noise mode indicator 25 may provide an indication to IMD 16 to revert to the alternative mode of operation via a predetermined electrical signal or signals, which IMD 16, e.g., sensing module 86 and processor 80, may detect via any of electrodes 40, 42, 44, 46, 48, 50, 58, 62, 64 and 66.

Power source 658 delivers operating power to the components of noise mode indicator 25. Power source 658 may include a battery and a power generation circuit to produce the operating power. In some examples, the battery may be rechargeable to allow extended operation. Recharging may be accomplished by electrically coupling power source 658 to a cradle or plug that is connected to an alternating current (AC) outlet. In addition or alternatively, recharging may be accomplished through proximal inductive interaction between an external charger and an inductive charging coil within noise mode indicator 25. In other examples, traditional batteries (e.g., nickel cadmium or lithium ion batteries) may be used. In addition, noise mode indicator 25 may be directly coupled to an alternating current outlet to power noise mode indicator 25. Power source 658 may include circuitry to monitor power remaining within a battery. In this manner, user interface 654 may provide a current battery level indicator or low battery level indicator when the battery needs to be replaced or recharged. In some cases, power source 658 may be capable of estimating the remaining time of operation using the current battery.

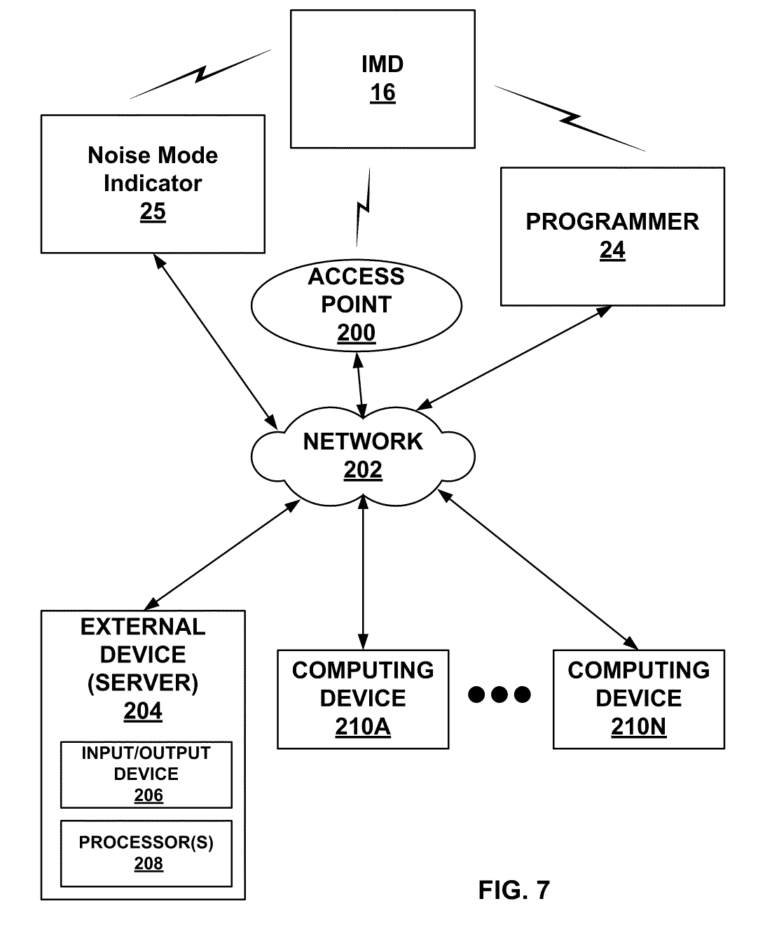

FIG. 7 is a block diagram illustrating an example system 190 that includes an external device, such as a server 204, and one or more computing devices 210A-210N, that are coupled to the IMD 16, programmer 24, and noise mode indicator 25 shown in FIG. 1 via a network 202. In this example, IMD 16 may use its telemetry module 88 to communicate with programmer 24 via a first wireless connection, to communication with an access point 200 via a second wireless connection, and to communicate with noise mode indicator 25 via a third wireless connection. In the example of FIG. 7, access point 200, programmer 24, noise mode indicator 25, server 204, and computing devices 210A-210N are interconnected, and able to communicate with each other, through network 202. In some cases, one or more of access point 200, programmer 24, noise mode indicator 25, server 204, and computing devices 210A-210N may be coupled to network 202 through one or more wireless connections. IMD 16, programmer 24, noise mode indicator 25, server 204, and computing devices 210A-210N may each comprise one or more processors, such as one or more microprocessors, DSPs, ASICs, FPGAs, programmable logic circuitry, or the like, that may perform various functions and operations, such as those described herein.

Server 204 and/or computing devices 210 may, for example, provide an indication to IMD 16, e.g., via network 202 and access point 200 or noise mode indicator 25, to revert to a noise mode of operation. In some examples, the server or computing device provides the indication at some time prior to when IMD 16 will experience the relatively high noise condition, and the indication will be in the form of an instruction to revert to the noise mode of operation at a future time coincident with the relatively high noise condition. For example, the server or computing device may instruct IMD 16 to revert to the noise mode of operation at a time in the future and for a period of time in which patient 14 and/or IMD 16 is scheduled to be in an operating room.

Access point 200 may comprise a device that connects to network 186 via any of a variety of connections, such as telephone dial-up, digital subscriber line (DSL), or cable modem connections. In other examples, access point 200 may be coupled to network 130 through different forms of connections, including wired or wireless connections. In some examples, access point 128 may be co-located with patient 14 and may comprise one or more programming units and/or computing devices (e.g., one or more monitoring units) that may perform various functions and operations described herein. For example, access point 200 may include a home-monitoring unit that is co-located with patient 14 and that may monitor the activity of IMD 16. In some examples, a home monitoring unit may relay to IMD 16 instructions to function in an alternative operating mode. In some examples, server 204 or computing devices 210 may perform any of the various functions or operations described herein.

Network 202 may comprise a local area network, wide area network, or global network, such as the Internet. The system of FIG. 7 may be implemented, in some aspects, with general network technology and functionality similar to that provided by the Medtronic CareLink® Network developed by Medtronic, Inc., of Minneapolis, Minn.

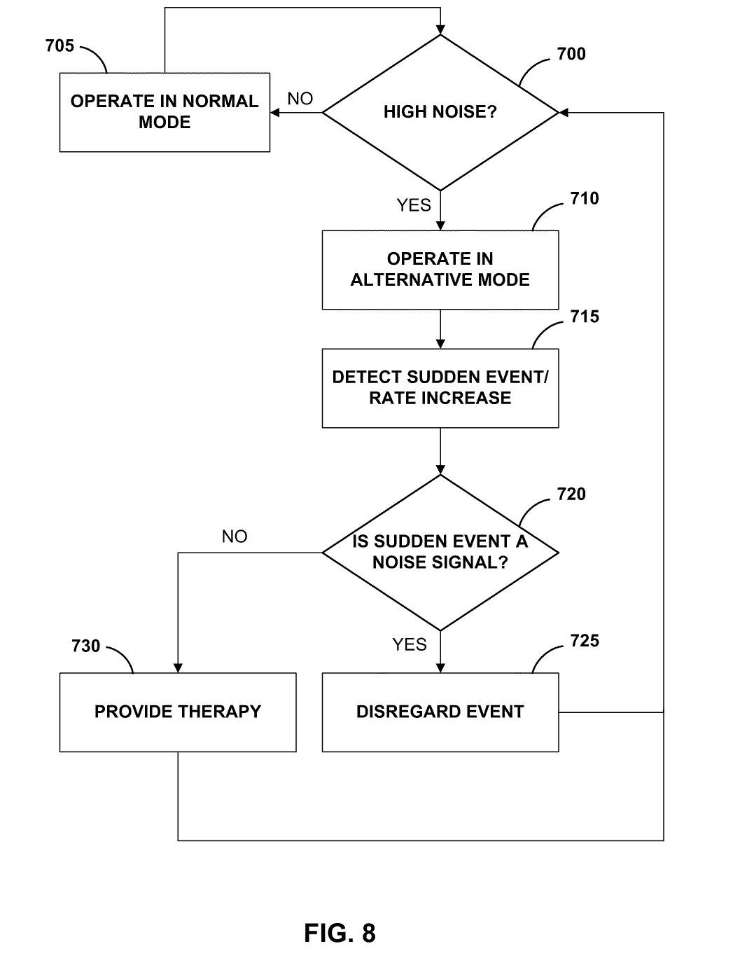

FIG. 8 is a flow diagram of an example method of operating an implantable medical device in an alternative operating mode when a relatively high level of noise is present. The functionality described with respect to FIG. 8 as being provided by a particular processor of device may, in other examples, be provided by any one or more of the processors or devices described herein.

An IMD 16 may make a determination as to whether it is experiencing high noise, e.g., in a high noise environment (700). For example, IMD 16 may analyze signals received from electrodes connected to sensing module 86 for evidence of noise, .e.g., based on a rate of events detected by narrow band channels 102, correlation of events across different channels when such correlation would not be physiologically expected, or an analysis of the morphology of the signal received via a wide band channel 104 for evidence of noise. In one example, the IMD 16 may periodically check for high levels of noise. The frequency of the check by the IMD may be a setting that a user may be able to input directly or via another device, for example, a programmer 24. The check may be triggered by an event that the IMD may consider unusual or out of the ordinary. For example, the check may be triggered by a tachyarrhythmia detection or an event that fulfills the criteria for the IMD 16 to deliver shock therapy.

In another example, the IMD 16 may receive an indication from another device such as, for example, noise mode indicator 25 that the IMD is in a high noise environment. For example, a user or clinician may use the noise mode indicator 25 to send a signal to the IMD that it is in a high noise environment. In another example, a user or clinician may program noise mode indicator 25 in advance to communicate to the IMD a future time during which the IMD is expected to be in a high noise environment, for example, when the IMD will be in an operating room. In some examples, the user or clinician may later use the noise mode indicator 25 to send a signal to the IMD that it is no longer in the high noise environment, and to exit the alternative operating mode and revert back to its normal operating mode.

If the IMD determines that there is not a high level of noise, the IMD may continue to operate in its normal mode (705). When in a relatively high noise condition, e.g., as indicated by a signal from a user or external device, the IMD may revert to an alternative operating mode (710).

In some examples, during operation in the alternative noise mode, IMD 16 (e.g., processor 80) may disable certain functionality of the IMD. For example, IMD 16 may suspend sensing by sensing module 86, analysis of the indications from sensing module 86 by processor 80 for the purpose of identifying arrhythmia, and/or delivery of therapy responsive to such analysis, e.g., delivery of defibrillation shocks. IMD 16 may also switch pacing modes, e.g., revert to asynchronous pacing.

In other examples, during the alternative operating mode, the IMD may analyze input signals differently to determine whether an input signal is a physiological signal that requires delivering therapy, such as, for example, VF/VT, or a noise signal that should be ignored. While the IMD is operating in the alternative operating mode it may detect a sudden event and/or a rate increase (715). During normal operating mode, the IMD may determine that a sudden event is a physiological signal, determine the channel through which the signal was detected, and deliver the appropriate therapy. For example, in normal operating mode, the IMD may sense a physiological signal that it may determine to be VF/VT, to which it may respond by delivering a shock to remedy the detected condition.

When the IMD is operating in the alternative operating mode and it detects a sudden event and/or a rate increase, it checks whether the sudden event is a noise signal (720). There are several ways the IMD may check whether an event is a noise signal, as will be discussed in more detail below. If the IMD determines that the signal is a noise signal, it may disregard it and do nothing (725). Otherwise, if the IMD determines that the event is indeed a physiological signal, then the IMD may deliver the appropriate therapy (730). Once the IMD makes a decision regarding the detected event, it may return to check whether it is still in the high noise condition, e.g., still detecting noise, or still under direction from a user or device to operate in the noise mode (700).

In one example, while operating in the alternative operating mode, the IMD may disregard a sudden event without checking whether the sudden event is a noise signal. For example, if the switch to the alternative operating mode was triggered by a user's instruction to operate in the alternative operating mode, sudden events may be disregarded without checking, and/or responsive therapy may be disabled. In another example, such as when the reversion to the alternative mode was triggered by detection of noise, the IMD may continue to monitor for and analyze high rate events, but use a more cautious event detection algorithm, e.g., with an increased NID and/or increased efforts to detect noise.

While operating in the alternative mode, if an event is determined to be most likely a noise signal, physiological event detection decisions may be withheld, until there is a determination that noise is no longer present. In an example, determining that noise is no longer present may be a function of confidence of recent noise presence. Therefore, if there is high confidence that noise was recently present, there may be an assumption that the environment is still noisy and less level of confidence may be needed to continue operating in a “assume sudden event is noise” mode. The level of confidence may depend on the how recent a sudden signal was detected as noise, where, for example, confidence of recent noise presence is higher if a noise signal was detected within the last several minutes, than if it was detected over a half hour ago, for example.

In some examples, when the IMD is in a high noise environment, the environmental noise will likely affect all sense channels. Therefore, if the IMD recognizes a sudden increase in the VF zone, the IMD may check other sensing channels, e.g., an atrial sense channel 102, 104, or a channel no presently coupled to any electrodes by switching module 100, to determine whether they have also sensed a fast rate and/or a similar sudden onset. In this example, the IMD may perform this determination by looking for short intervals, i.e., sensed intervals just outside blanking. If other channels have also sensed a fast rate and/or a similar sudden onset, the event is likely a noise event and the IMD may disregard it. Otherwise, the event is a physiological signal that requires therapy and the IMD may proceed accordingly.

In another example, a sense channel that may be used to detect physiological events during normal operating mode, may be repurposed to operate in an alternative manner during the IMD's alternative operating mode. The sense channel may be a channel used generally for capture detection, which may be run periodically, for example, once a day, and if it detects a fast rate, the capture detection may be disabled. During the alternative operating mode, if a fast rate is detected, the sense channel for capture detection may be enabled to detect a noise signal, without directly connecting the channel to any electrodes. If the IMD detects a sudden event and/or a fast rate signal, noise may be suspected. If the capture detection channel senses a cluster of events, decisions regarding physiological event detection, such as VT/VF detection may be withheld, until normal signal behavior is sensed by the capture detect channel.

In another example, when the IMD is in a high noise environment, and it detects a sudden event, the IMD may use morphology analysis of the sensing channels (or any EGM channel) to determine whether the event is a physiological or noise signal. The IMD may perform morphology analysis of a window around the sensed events on any EGM channel to determine whether the pattern is consistent with patterns of cardiac events or noise. Alternatively, the IMD may perform spectral analysis of the EGM channels over a larger window. If there are high frequency artifacts in the spectral analysis of the EGM channels, then the signal is likely a noise signal and the IMD may disregard it. The IMD may perform morphology and/or the spectral analyses on multiple channels to increase the confidence in the diagnosis of the event, and the determination as to whether it is a noise signal or not.

During normal operating mode, the IMD may look at a smaller number of intervals to make a decision regarding a detected event. In one example, the number of intervals to detect (NID) may be increased during the alternative operating mode, e.g., when the IMD is in an environment with higher levels of noise. The IMD may then reset the NID to its normal smaller value, when the IMD is no longer in the alternative operating mode. For example, referring to FIG. 7, when the IMD begins operating in the alternative mode (710), or when a noise signal is detected while operating in the alternative mode, the NID may be increased, and the detection decision process may be altered so that events indicating noise signals may be disregarded. The IMD may be restored to its normal value, when the IMD is also returned to normal operating mode.

In another example, a control device, e.g., noise mode indicator, may be placed in a high noise environment where a patient may at some time be present with an IMD. The high noise environment may be a room where there may be several machines that create interfering field, or an operating room, etc. The control device may be used to temporarily place the IMD in the alternative noise mode when the IMD is in the high noise environment, and return it to the normal operating mode when the cause of high noise signals is removed or the IMD leaves the high noise environment. The control device may communicate with the IMD through telemetry. In one example, the control device may be set up by a user to turn off detection on an IMD for a specified amount of time if the patient with the IMD is going to be in the high noise environment for a specified amount of time, in which case, an input from the user may turn off the detection on the IMD, and after the specified amount of time, the IMD detection may turn back on automatically. High noise environments such as, for example, operating rooms, may be equipped with the control device, which may be a simple on/off button, and may also have an indicator that provides the user an indication regarding the current state of detection, i.e., whether detection is on or off.

In another example, the IMD may be programmed with a time during which the patient is scheduled to be in a high noise environment, such as, for example, an operating room. A user may program the date of the surgery, for example, to the IMD using a programmer or the date may be down linked remotely through a system, such as, for example, CareLink, to a home monitor, and to the device. The IMD may be that way programmed to automatically switch to the alternative operating mode where detection may be turned off on the specified date/time. While in that mode, the IMD may operate as described above, where sudden events are diagnosed with care and disregarded if determined to be noise signals and not physiological events.

Although the disclosure is described with respect to cardiac stimulation therapy, such techniques may be applicable to other therapies in which sensing integrity is important, such as, e.g., spinal cord stimulation, deep brain stimulation, pelvic floor stimulation, gastric stimulation, occipital stimulation, functional electrical stimulation, and the like.

The techniques described in this disclosure, including those attributed to image IMD 16, programmer 24, or various constituent components, may be implemented, at least in part, in hardware, software, firmware or any combination thereof. For example, various aspects of the techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, image processing devices or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic data storage media, optical data storage media, or the like. The instructions may be executed to support one or more aspects of the functionality described in this disclosure.

Various examples have been described. These and other examples are within the scope of the following claims.

1. A method comprising:
receiving in an implantable medical device a signal from an external control device, wherein the signal comprises an instruction from a user to the implantable medical device to revert to a predetermined operating mode for a noise condition; and
reverting the implantable medical device to the predetermined operating mode in response to the signal, wherein reverting to the predetermined operating mode comprises modifying at least one of physiological event detection or delivery of therapy in response to physiological event detection by the implantable medical device.

2. The method of claim 1, wherein modifying at least one of physiological event detection or delivery of therapy in response to physiological event detection comprises disabling at least one of physiological event detection or delivery of therapy in response to physiological event detection.

3. The method of claim 1, wherein modifying physiological event detection comprises:
detecting a rate event resembling a physiological event on at least a first sensing channel associated with the implantable medical device;
determining based on signals received via at least a second sensing channel associated with the implantable medical device whether the rate event is a false physiological event; and
disregarding the rate event if it is a false physiological event.

4. The method of claim 3, wherein determining via at least the second sensing channel whether the rate event is a false physiological event comprises comparing the event detection via the at least first sensing channel to the event detection via the at least second sensing channel.

5. The method of claim 3, wherein determining via at least the second sensing channel whether the rate event is a false physiological event comprises analyzing a morphology of a signal received via the at least second sensing channel.

6. The method of claim 5, wherein analyzing the morphology comprises comparing the rate event detected via the second sensing channel to templates of false physiological events.

7. The method of claim 1, wherein modifying physiological event detection comprises modifying a number of intervals to detect for tachyarrhythmia detection.

8. The method of claim 1, wherein the signal comprises a beacon that is delivered one of periodically or substantially constantly, the method further comprising:
determining, by the implantable medical device, cessation of the signal; and
exiting the predetermined operating mode in response to the determination.

9. The method of claim 1,
wherein the implantable medical device comprises at least one of a pacemaker, a cardioverter, or a defibrillator, and
wherein modifying at least one of physiological event detection or delivery of therapy in response to physiological event detection comprises modifying at least one of detection of cardiac depolarizations, detection of tachyarrhythmias, delivery of cardiac pacing, delivery of cardioversion, or delivery of defibrillation.

10. A system comprising an implantable medical device, wherein the implantable medical device comprises:
an electrical sensing module for detection of physiological events;
circuitry that receives a signal from an external control device, wherein the signal comprises an instruction from a user to the implantable medical device to revert to a predetermined operating mode for a noise condition;
a processor that reverts the implantable medical device to the predetermined operating mode in response to the signal by at least modifying at least one of physiological event detection or delivery of therapy by the implantable medical device in response to physiological event detection by the implantable medical device.

11. The system of claim 10, wherein the processor disables at least one of physiological event detection or delivery of therapy in response to physiological event detection for operation in the predetermined operating mode.

12. The system of claim 10,
wherein the sensing module comprises a first sensing channel that detects a rate event resembling a physiological event and a second sensing channel, and
wherein the processor determines based on signals received via the second sensing channel whether the rate event is a false physiological event, and disregards the rate event if it is a false physiological event.

13. The system of claim 12, wherein the processor compares the event detection via the at least first sensing channel to the event detection via the at least second sensing channel to determine whether thee rate event is the false physiological event.

14. The system of claim 13, wherein the processor analyzes a morphology of a signal received via the at least second sensing channel to determine whether thee rate event is the false physiological event.

15. The system of claim 14, wherein the processor compares the rate event detected via the second sensing channel to templates of false physiological events to determine whether thee rate event is the false physiological event.

16. The system of claim 10, wherein the processor modifies a number of intervals to detect for tachyarrhythmia detection for operation in the predetermined operating mode.

17. The system of claim 10, wherein the signal comprises a beacon that is delivered one of periodically or substantially constantly, the circuitry determines cessation of the signal, and the processor exits the predetermined operating mode in response to the determination.

18. The system of claim 10, wherein the processor reverts the implantable medical device to the predetermined operating mode at an indicated future time in response to the signal

19. The system of claim 10,
wherein the implantable medical device comprises at least one of a pacemaker, a cardioverter, or a defibrillator, and
wherein the processor modifies at least one of detection of cardiac depolarizations, detection of tachyarrhythmias, delivery of cardiac pacing, delivery of cardioversion, or delivery of defibrillation for the predetermined operating mode.

20. The system of claim 10, further comprising the control device, wherein the control device delivers the signal in response to a single user input.

21. The system of claim 10, further comprising the control device, wherein the control device delivers the signal in response to powering on of the control device.

22. The system of claim 10, further comprising the control device and an external programmer, wherein interaction with the implantable medical device by control device is limited with respect to interaction with the implantable medical device by the external programmer.

23. A system comprising:
means for receiving in an implantable medical device a signal from an external control device, wherein the signal comprises an instruction from a user to the implantable medical device to revert to a predetermined operating mode for a noise condition; and
means for reverting the implantable medical device to the predetermined operating mode in response to the signal, wherein the means for reverting to the predetermined operating mode comprises means for modifying at least one of physiological event detection or delivery of therapy in response to physiological event detection by the implantable medical device.

24. A computer readable storage medium comprising instructions that cause a processor of an implantable medical device to:
receive a signal from an external control device, wherein the signal comprises an instruction from a user to the implantable medical device to revert to a predetermined operating mode for a noise condition; and
revert the implantable medical device to the predetermined operating mode in response to the signal, wherein the instructions that cause the processor to reverting to the predetermined operating mode comprise instructions that cause the processor to modify at least one of physiological event detection or delivery of therapy in response to physiological event detection by the implantable medical device.
